(19) United States
(12) Patent Application Publication
(10) Pub. No.: US 2017/0165060 A1
(43) Pub. Date: Jun. 15, 2017

(54) CUTTING STENT ASSEMBLY INCLUDING PROSTHETIC COMPONENT

(71) Applicant: Mark Gelido Barongan, San Diego, CA (US)

(72) Inventor: Mark Gelido Barongan, San Diego, CA (US)

(21) Appl. No.: 15/376,417

(22) Filed: Dec. 12, 2016

Related U.S. Application Data
(60) Provisional application No. 62/265,494, filed on Dec. 10, 2015.

(51) Int. Cl. A61F 2/24

(52) U.S. Cl. CPC A61F 2/2418; A61F 2220/0016

(57) ABSTRACT

A cutting stent device including a prosthetic component positioned within the central lumen of the stent. The stent device includes blades with cutting edges and beveled ends as disclosed and described in U.S. Pat. No. 8,876,882 issued Nov. 4, 2014, incorporated herein by reference in its entirety, The prosthetic component may be a tissue valve comprising bovine or porcine pericardium tissue. The cutting stent device including prosthetic component reduces, if not eliminates, “para-valvular leaking” as caused by mal-apposition. The stent device according to invention provides full apposition between the prosthetic component and native valve leaflets and annulus.

### CROSS REFERENCE TO RELATED PATENTS

This application claims the benefit of U.S. Provisional Patent Application No. 62/265,494 filed Dec. 10, 2015, which is hereby incorporated by reference in its entirety.

### FIELD OF THE INVENTION

The invention relates generally to a medical surgical device and more specifically to a stent device comprising blades that cut and embed, or wedge, into an anatomical structure to alleviate, or possibly eliminate an abnormality when used in an anatomical structure, for example, a blood vessel, body cavity, orifice, chamber, or organ to provide structural support. More specifically, the invention is directed to an assembly comprising a cutting stent device and a prosthetic component such as a tissue valve.

### BACKGROUND OF THE INVENTION

Many different medical procedures are performed each year. Due to rising healthcare costs, it is desirable to minimize time and equipment related to each procedure in an effort to off-set these costs. For example, a traditional surgical procedure to replace a calcified heart valve on a patient may cost nearly U.S. $200,000. There is a constant need for improved medical devices that work as effectively, if not better, than traditional devices and that also reduce cost.

One type of medical device used regularly is an intravascular stent. Stent devices are commonly used to maintain patency, opening or spread, of a blood vessel in an attempt to maintain normal blood flow in vessels for various situations. These situations include, for example, stenosis, constriction of vessels due to plaque, calcium, thrombus, and other debris, or combination of these. Intravascular stents are typically metallic and self-expanding or balloon expandable. Certain stent devices are designed to deploy in a circumferential shape and may be mounted on a delivery-type catheter. Many stent devices are designed with struts and have an opened-cell or repeating-cell design.

Newer methods directed to prosthetic components, such as trans-catheter valve repair, are being used in certain procedures resulting in reduced cost and shorter hospital stays for patients. As an example, prosthetic components are used in heart valve replacement procedures.

Heart valves—mitral, tricuspid, aortic, pulmonary—comprise thin flaps of tissue called leaflets and an annulus, a tough fibrous ring attached to the leaflets that help support and maintain the proper shape of the valve. During heart valve replacement procedures, a prosthetic valve is loaded onto a catheter and expanded into the patient's native valve. In the case of a prosthetic heart valve, a stent device is used along with a prosthetic valve. The prosthetic valve is secured within the interior of the stent device. In certain embodiments, the prosthetic valve is secured to the stent device using sutures. When the stent is implanted and the prosthetic valve deployed, the stent device expands into the calcific native heart valve and the leaflets of the prosthetic valve replace the patient's old native leaflets. The old native leaflets are crushed against the native annulus by the stent device. It is the calcific nature of the native leaflets and native annulus that help position and affix the prosthetic valve in place.

When deployed, it is desirable that the prosthetic valve be apposed against the native valve. However, sometimes the native valve tissue and native valve annulus do not fully appose the prosthetic valve such as in situations of atherosclerosis. In particular, atherosclerosis may cause thickening of the native valve transforming it from its ideal round cross-sectional shape to an angular or oval shape. Furthermore, the native valve and deployed prosthetic valve may not be congruent leaving a communication gap between the prosthetic valve and native valve including leaflets.

With many prosthetic implantation procedures, there is a phenomenon known as “para-valvular leaking.” This occurs when the prosthetic device is not fully apposed to both the native leaflets and valve annulus, referred to as “mal-apposition”. Mal-apposition may prevent the prosthetic leaflets of the prosthetic valve from being fully taut once deployed.

Additional dilation may be performed if para-valvular leaking exists, but doing so does not always prove effective for apposition and may further result in an adverse outcome, such as native valve annulus tearing, tamponade, and/or induced arrhythmia, some of which require an additional procedure such as a permanent pacemaker.

To verify functionality and fitment of an implanted prosthetic valve, transesophageal or transthoracic echocardiography is typically used. However, the prosthetic valve cannot be fully assessed for para-valvular leaking using echocardiography until the prosthetic valve is implanted and fully deployed.

What is needed is a cutting stent device including prosthetic component that mitigates “para-valvular leaking” caused by mal-apposition of the prosthetic component with the native valve. The present invention satisfies this need.

### SUMMARY OF THE INVENTION

The stent assembly according to the invention comprises a stent device and a prosthetic component. The stent device includes blades with cutting edges and beveled ends such as those as described U.S. Pat. No. 8,876,882 issued Nov. 4, 2014, incorporated herein by reference in its entirety.

More specifically, the stent device comprises a body with blades that are longitudinally configured at equidistant points along the direction of the stent body. For example, a first pattern has five blades equidistant from the other and running longitudinally along the direction of the stent body. Another pattern has five shorter blades equidistant from the other to constitute one set. Each set is arranged in a staggered pattern from the next set.

Each blade includes a cutting edge positioned between beveled ends, with each cutting edge being linear and oriented substantially parallel to a longitudinal axis centered in a lumen of the body. The ends of the blades are angular or beveled to facilitate the entry of the stent device into an anatomical structure. The beveled ends assist in the entrance and placement of the stent device into the anatomical structure upon expansion and implantation of the stent into the anatomical structure. The cutting edges disposed on the blade cut and wedges the stent in the anatomical structure. Each cutting edge creates one or more fissures and scores in the anatomical structure in a direction of the longitudinal axis.

The prosthetic component is disposed within the central lumen of the stent. The prosthetic component may be any type of prosthetic valve, for example, pyloric and ileocecal valves, upper and lower esophageal sphincter valves, chambers, organs, lumens including trans-thoracic lumens, ducts including excretion ducts, canals, pockets (subcutaneous), and fistulas, to name a few.

According to one embodiment, the prosthetic component is a heart valve, for example, constructed of two or more leaflets and a valve body portion. The valve is preferably a tissue valve comprising bovine or porcine pericardium tissue. The cutting stent device and/or prosthetic component may also be coated in a biocompatible material, as well as with other biomolecules such a therapeutic agent.

The cutting stent device including prosthetic component may reduce the possibility of cutting into ancillary anatomical structures, such as an artery, caused by over-inflation of the current cutting balloon. Using the current cutting balloon causes micro tears on the intimal wall of an anatomical structure. Additionally, the chances of over-inflating, causing a serious score through the anatomical structure, are reduced by the stent pattern with integrated blades.

The cutting stent device including prosthetic component addresses the issues of mal-apposition and para-valvular leaking. The blades with cutting edges and beveled edges provide better fitment with the native valve annulus minimizing, if not eliminating, mal-apposition and para-valvular leaking.

The cutting stent device including prosthetic component reduces the multi-step process that requires the exchange of a post dilatation balloon to place in the stent with prosthetic component. Generally, the less number of steps involved in an intravascular procedure, the safer and better the procedure is considered. Multi-step processes can elevate the complication and chances of scratching of intimal wall, cuts, punctures, falling debris, and the like, which ultimately can lead to more serious problems and complications. Therefore, cutting stent device including prosthetic component reduces para-valvular leaking and ensure implant apposition the likelihood of plaque and debris that can be dislodged distally causing flow obstruction.

The cutting stent device including prosthetic component reduces disruption of debris that may be caused by scoring of the hard plaque and debris. The cutting stent device including prosthetic component has angular or beveled faces at the beginning and end of each blade. This angular raise facilitates the ease of positioning the blades into to plaque and debris. With fewer disturbances in the area, the likelihood of falling debris from the plaque also decreases.

The other cause of falling debris in the blood stream is the multiple contacts of the intravascular procedural equipment, The cutting stent device including prosthetic component eliminates the need to change equipment in and out of an anatomical structure like the changing of equipment if a cutting balloon were used since the cutting blades serve the function as the cutting balloon.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

The preferred embodiments of the invention will be described in conjunction with the appended drawings provided to illustrate and not to the limit the invention, where like designations denote like elements, and in which:

FIG. 1A illustrates a cutting stent device according to the invention.

FIG. 1B illustrates another cutting stent device according to the invention.

FIG. 2 illustrates a perspective view of a blade of the cutting stent device according to the invention.

FIG. 3 illustrates a top view of a blade of the cutting stent device according to the invention.

FIG. 4 illustrates an assembly comprising a stent device and a prosthetic component according to the invention.

### DETAILED DESCRIPTION OF EMBODIMENTS OF THE INVENTION

The present invention incorporates by reference in its entirety U.S. Pat. No. 8,876,882 issued Nov. 4, 2014. In addition to the intravascular procedure discussed in the '882 Patent, the invention is applicable to other human anatomy such as the digestive system, urinary system, reproductive system, skeletal system, respiratory system, muscular system, lymphatic system, nervous system, and immune system. For example, embodiments of the invention may be used with an anatomical structure in the form of blood vessel, valves (e.g. pyloric and ileocecal valves, and the upper and lower esophageal sphincter valves, heart valve such as the mitral valve, tricuspid valve, aortic valve, or pulmonary valves), chambers, organs, lumens including trans-thoracic lumens, ducts including excretion ducts, canals, pockets (subcutaneous), and fistulas, to name a few.

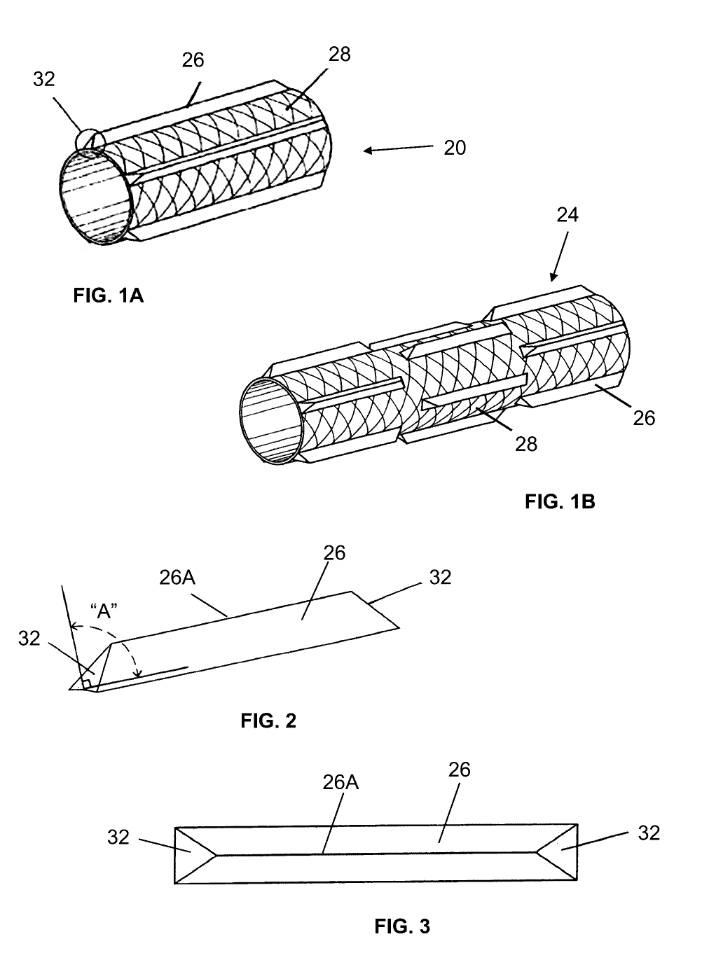

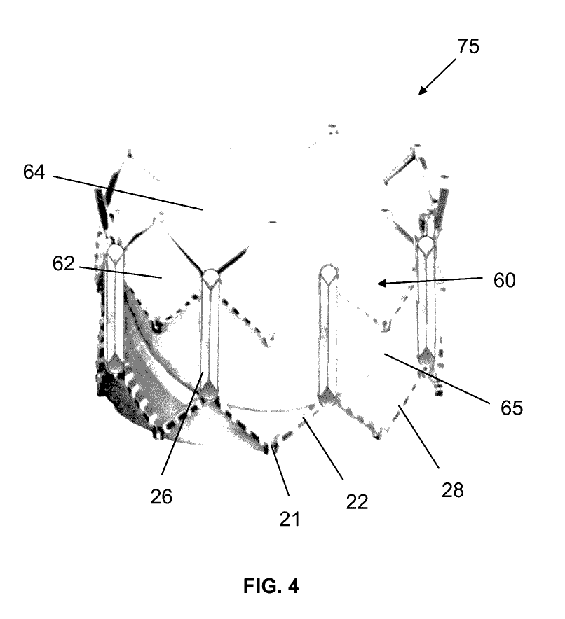

With reference to FIG. 1A and FIG. 1B, a stent device 20, 24 comprises struts 28 and blades 26. As shown in FIG. 1A, the stent device 20 includes a non-staggered pattern of blades 26. A staggered pattern of blades 26 is shown in the stent device 24 of FIG. 1B. Each stent device includes a plurality of struts 28 with each strut 28 including an open ring 21 and an arm 22 extending from the open ring 21, as shown in FIG. 4. Another open-end circle, or open ring 21, joins at the end of each arm 22. The pattern of open-ended circles and arms replicate to form an expandable, flexible pattern that integrates each blade 26.

FIG. 2 and FIG. 3 illustrate a blade 26 of the cutting stent device. As shown, each blade 26 comprises a first free end extending to a second free end, wherein each end is beveled forming a beveled end 32. Each beveled end is at an acute angle with respect to the base of the blade. The preferred angle “A” for the end surface of blade 26 is somewhere between 5 degrees to 45 degrees. This beveled edge 32 facilitates ease in crossing blockages with low resistance and assists the stent device to ease into place. More specifically, the beveled ends assist in the entrance and placement of the stent device into the native valve upon expansion and implantation of the stent into the valve to be replaced. However, it is contemplated the angle can range between 1 degree to 80 degrees.

Each blade 26 includes a cutting edge 26A. The cutting edge 26A is disposed on blade 26 positioned between beveled edges 32. The cutting edges disposed on the blade cut into the blockage to wedge the stent in the native valve comprising plaque, calcium, and other calculus debris.

The stent material may comprise any conventional metal such as stainless steel, titanium, nickel titanium, Nitinol, tantalum, gold, cobalt-chromium, platinum, palladium, iridium, or other metals, metal alloys, metalloids and bio-absorbable polymers, polymers may also be used to comprise the stent device.

Stents may also be constructed of polymers consisting of polyurethanes, polyetherurethanes, polyesterurethanes, silicone, thermoplastic elastomer (C-flex), polyether-amide thermoplastic elastomer (Pebax), fluoroelastomers, fluorosilicone elastomer, styrene-butadiene rubber, butadiene-styrene rubber, polyisoprene, neoprene (polychlor prene), ethylene-propylene elastomer, chlorosulfonated polyethylene elastomer, butyl rubber, polysulfide elastomer, polyacrylate elastomer, nitrile rubber, a family of elastomers composed of styrene, ethylene, propylene, aliphatic polycarbonate polyurethane, polymers augmented with antioxidants, polymers augmented with image enhancing materials, polymers having a proton (H+) core, polymers augmented with protons (H+), butadiene and isoprene (Kraton®) and polyester thermoplastic elastomer (Hytrel®), polyethylene, PLA, PGA, and PLGA.

The blades may be constructed of the same or different material then the stent body on which they are disposed. For example, the blades may be constructed of cobalt nickel steel, stainless steel, glass materials, ceramic materials, and biodegradable materials, In some embodiments, the blades may be constructed of Nitinol or other memory shape material.

With reference to FIG. 4, shown is one embodiment of the assembly 75 for use with a prosthetic component 60. Although the prosthetic component is discussed with respect to a tissue valve, any anatomical structure is contemplated for which a prosthetic component may be necessary, for example, blood vessel, chambers, organs, lumens including trans-thoracic lumens, ducts including excretion ducts, canals, pockets (subcutaneous), and fistulas, to name a few.

More particularly, the invention is described with respect to a heart tissue valve, but any prosthetic valve is contemplated, for example, pyloric and ileocecal valves, upper and lower esophageal sphincter valves, chambers, organs, lumens including trans-thoracic lumens, ducts including excretion ducts, canals, pockets (subcutaneous), and fistulas, to name a few.

The prosthetic component 60 comprises a valve body portion 62 and two or more leaflets or cusps 64. The body portion 62 is positioned within the lumen of the stent body. A valve opening is generally located within the center of the leaflets 64 through which fluid flows. A prosthetic valve may comprise any number of leaflets, such two or more leaflets, at least three leaflets, at least four leaflets, etc. The valve leaflets are configured to collapse inwardly (i.e., towards the central longitudinal axis) inhibit retrograde fluid (e.g. blood) flow, and to open outwardly to allow fluid flow through the prosthetic component 60.

The prosthetic component 60 may be connected to the stent device 20, 24 using any attachment means known in the art, for example adhesive, thread, or as shown in FIG. 4 a suture 65. Other contemplated attachment means may include radiofrequency adhesion or chemical bonding. The number of sutures attaching the prosthetic component to the stent device may vary. An embodiment of the present invention may comprise a single continuous suture, or any number of smaller sutures.

In a preferred embodiment, sutures connect the valve body 62 to the struts 28 of the stent device 20, 24 at any suitable position. For example, the valve body 62 may be connected to the open rig 21 and/or the arm 22 of one or more struts 28. It is also contemplated that the valve body 62 is connected to the stent device 20, 24 using an attachment means at or near the blades 26, including for example at or near the beveled edges 32.

A prosthetic valve may be constructed of a natural biocompatible material or synthetic material, or a combination thereof.

Examples of biocompatible material for use as leaflets or cusps, includes but is not limited to, porcine, bovine, or equine pericardial tissue, stomach submucosa, collagen, liver basement membrane, urinary bladder submucosa, tissue mucosa, dura mater, and small intestine submucosa. Synthetic materials include polyethylene terephalate(PET), polypropylene(PP), polytetrafluorethylene(PTFE), or any polymer or derivative thereof, and also includes commercially known materials such as GORE-TEX®, DACRON®, polyglactin, copolymers of lactide and caprolactone, and polylactides, synthetic fabrics such as knit or woven polyester, and non-woven fabrics, and collagen-impregnated fabrics. In some embodiments, the valve material can be made thicker by making multi-laminate constructs.

The assembly 75 is positioned into the native valve by first using a supportive guidewire. A The guidewire is inserted in the same fashion to extend through the distal end of the native valve. Next, a stent assembly 75 is inserted into a guiding sheath catheter. Then it is traversed over the guidewire to the native valve. The stent assembly is mounted on an expandable balloon and passed over the guidewire into the native valve. Once the stent assembly slides through the native valve, the balloon is inflated to a nominal atmospheric pressure by use of a balloon inflation device. While the stent device is being inflated, the stent blades cut and wedge into the native valve annulus of the native valve. When the stent assembly is implanted and the prosthetic valve deployed, the stent device expands into the calcific native heart valve and the leaflets of the prosthetic valve replace the patient's old native leaflets. The old native leaflets are crushed against the native annulus by the stent device. It is the calcific nature of the native leaflets and native annulus that help position and affix the prosthetic valve in place. When deployed, the prosthetic valve is apposed against the native valve. More specifically, the native valve and deployed prosthetic valve are congruent leaving no communication gap between the prosthetic valve and native valve including leaflets.

In other embodiments of the inventions, the stent device 20, 24 and/or prosthetic component 60 may be coated with an organic material such as a biocompatible polymer, other biomolecules.

The biocompatible material should minimize irritation to a lumen wall when the device is implanted. Such materials include, for example, poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphazenes. Also, biostable polymers with a relatively low chronic tissue response such as polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Exemplary biomolecules include fibrin, fibrinogen, cellulose, starch, collagen and hyaluronic acid.

In further embodiments of the invention, the stent device 20, 24 and/or prosthetic component 60 may be coated with a therapeutic agent. Examples of therapeutic agents include: actinomycin D, or derivatives and analogs thereof, paclitaxel, docetaxel methotrexate, azathioprine, vincristine, vinblastine, fluorouracil, doxorubicin hydrochloride, mitomycin, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIa platelet membrane receptor antagonist antibody, recombinant hirudin, angiopeptin, cilazapril or Lisinopril, calcium channel blockers, colchicine, fibroblast growth factor (FGF) antagonists, fish oil (omega 3-fatty add), histamine antagonists, lovastatin, monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine, alpha-interferon, genetically engineered epithelial cells, nucleic acid, enzymes, tacrolimus, dexamethasone, and rapamycin and structural derivatives or functional analogs thereof, permirolast potassium and any combination thereof.

In further embodiments of the invention, the assembly 75 may comprise radiopaque agents such as tantalum, barium, bismuth, or the like to increase radiopacity. These agents may be rubbed, bonded or adhered to stent device 20, 24 and/or prosthetic component 60 to assist in positioning the assembly 75 using radiographic visualization or similar means during the installation procedure.

It is further contemplated that the assembly may include one or more biosensors, for example nano-sensors, to monitor blood chemistry, pH, Proteins, cell related markers, etc, and communication the monitored results such as through a computer program including, for example, an application for portable handheld devices or tablets.

In certain embodiments, the biosensors may be integrated into the prosthetic component, such as by woven into, within, on, or around the prosthetic component. It is also contemplated that the biosensors may be fused or attached to the stent device, such as the blades, beveled ends, struts. The biosensor may also be blended within the composition of the material used to make all or a portion of the stent device.

While the disclosure is susceptible to various modifications and alternative forms, specific exemplary embodiments of the present invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

1. A stent assembly, comprising:
a stent device including:
a plurality of struts forming a stent body, the plurality including a space between two struts;
one or more blades, wherein each blade includes a base with a first edge and a second edge, the first edge and the second edge attached to two or more struts such that each of the one or more blades are integral with said plurality of struts with the base extending over a portion of the space between two struts, each blade further comprising:
a first free end extending to a second free end, wherein the first free end is beveled forming a first beveled end and the second free end is beveled forming a second beveled end, the first beveled end and the second beveled end each at an acute angle with respect to the base of the blade;
a cutting edge disposed on the blade, wherein the cutting edge is positioned between the first beveled end and the second beveled end and the cutting edge is distally opposed to the base of the blade, wherein the cutting edge of the blade is linear and oriented substantially parallel to a longitudinal axis centered in a lumen of the stent body,
wherein the longitudinal axis centered in the lumen of the stent body extends from a proximal opening of the stent body to a distal opening of the stent body, and
wherein the first beveled end and the second beveled end of the blade is configured for entrance into the blockage of the blood vessel upon expansion and implantation of the stent into the blood vessel and the cutting edge disposed on the blade is configured to cut into the blockage to wedge the stent in the blood vessel; and
a prosthetic component attached within the lumen of the stent device.

2. The stent assembly of claim 1, wherein the prosthetic component is a tissue valve

3. The stent assembly of claim 1, wherein the prosthetic valve comprises a valve body portion and at least two leaflets.

4. The stent assembly of claim 1, wherein the prosthetic component is sutured to the stent device.

5. The stent assembly of claim 1, wherein the stent device is coated with at least one biocompatible material.

6. The stent assembly of claim 5, wherein the biocompatible material further comprises a biomolecule.

7. The stent assembly of claim 3, wherein the at least two leaflets are constructed of pericardium tissue.

8. The stent assembly of claim 1, wherein the stent device is coated with at least one therapeutic agent.
